(19) United States
(12) Patent Application Publication
(10) Pub. No.: US 2026/0283847 A1
(43) Pub. Date: Sep. 24, 2026

(54) AN OPHTHALMIC SURGERY INSTRUMENT

(71) Applicant: D.O.R.C. Dutch Ophthalmic Research Center (International) B.V., Zuidland (NL)

(72) Inventors: Pieter MOLENAAR, Zuidland (NL); Jürg ATTINGER, Zuidland (NL)

(21) Appl. No.: 19/473,616

(22) PCT Filed: Apr. 12, 2024
(86) PCT No.: PCT/NL2024/050191

§ 371 Date: Oct. 8, 2025

(30) Foreign Application Priority Data
Apr. 12, 2023 (NL) 2034561
Apr. 13, 2023 (NL) 2034566

(51) Int. Cl. A61F 9/007; A61B 17/28

(52) U.S. Cl. CPC A61F 9/007; A61B 17/2812

(57) ABSTRACT

The invention relates to an ophthalmic surgery instrument, comprising a handling unit, a surgical module provided on the handling unit, and a shaft at least partially surrounding the surgical module, wherein the surgical module and the shaft are mutually movable relative. The handling unit further comprises a guiding element arranged at a distal end of the handling unit. The handling unit comprises an actuation unit having a plurality of actuation arms, and a driving unit for driving the shaft relative to the guiding element back and forth through the central bore of the guiding element, wherein the driving unit includes a set of pivoting joint elements having ends that are pivotably connected to corresponding actuation arms.

### SUMMARY OF THE INVENTION

The invention relates to an ophthalmic surgery instrument, comprising a handling unit having a longitudinal axis, a surgical module provided on the handling unit and extending away therefrom along the longitudinal axis thereof, and a shaft at least partially surrounding the surgical module, wherein the surgical module and the shaft are mutually movable relative along the longitudinal axis of the handling unit, a guiding element arranged at a distal end of the handling unit, the guiding element being provided with a central bore that is traversed by the surgical module and the shaft extending from the handling unit outwardly along the longitudinal axis thereof, wherein the handling unit comprises an actuation unit comprising a plurality of actuation arms arranged around the longitudinal axis of the handling unit, and a driving unit arranged to, upon manual actuation of the actuation unit, drive the shaft relative to the guiding element back and forth through the central bore of the guiding element.

The above ophthalmic surgery instrument is known for example for ophthalmic surgery in the interior of the eye wherein the surgical module includes a movable distal portion such as a forceps. By providing a shaft partially surrounding the surgical module such that the shaft and the surgical module are movable relative to each other, the movable distal portion can selectively at least partly be covered or closed, or exposed or opened by the shaft, e.g. for actuating the surgical module and/or for inserting or removing the surgical module from the eye.

There is an ongoing need to improve manual operation of the ophthalmic surgery instrument.

It is an object of the present invention to provide an ophthalmic surgery instrument having improved manual operation characteristics. Therefore, according to the invention, an ophthalmic surgery instrument is provided, comprising a handling unit having a longitudinal axis, a surgical module provided on the handling unit and extending away therefrom along the longitudinal axis thereof, and a shaft at least partially surrounding the surgical module, wherein the surgical module and the shaft are mutually movable relative along the longitudinal axis of the handling unit, a guiding element arranged at a distal end of the handling unit, the guiding element being provided with a central bore that is traversed by the surgical module and the shaft extending from the handling unit outwardly along the longitudinal axis thereof, wherein the handling unit comprises an actuation unit comprising a plurality of actuation arms arranged around the longitudinal axis of the handling unit, the actuation arms each having a proximal end and a distal end, and a driving unit arranged to, upon manual actuation of the actuation unit, drive the shaft relative to the guiding element back and forth through the central bore of the guiding element, the driving unit including a radial-to-axial linkage constructed and arranged to convert a radial movement provided by the actuation unit through the actuation arms to axial movement of the shaft with respect to the surgical module, wherein the radial-to-axial linkage comprises a set of pivoting joint elements, each pivoting joint element having a first end and a second end, said second end being associated with the shaft, wherein the number of pivoting joint elements corresponds with the number of actuation arms, and wherein the first end of each pivoting joint element is pivotably connected to a corresponding actuation arm.

By providing the driving unit with a radial-to-axial linkage comprising a set of pivoting joint elements having first and second ends, the first ends being pivotably connected to corresponding arms and the second ends being associated with the shaft, operation of the instrument can be improved, e.g. in that operation of the shaft can be performed easily and reliably, based on a robust design.

Advantageously, the first end of the pivoting joint element is connected to the corresponding actuation arm so as to transfer both a pushing force from the actuation arm and a pulling force from the pivoting joint element, such that the actuation arms move as an ensemble. The pivotable connection can e.g. be realized using an axle bearing mechanism or a hinge structure.

One or more of the pivoting joint elements may be arranged such that its second end moves along the longitudinal axis of the handling unit upon adjusting the actuation arms from the decompressed state towards the compressed state.

Preferably, these pivoting joint elements are arranged at locations that are proximally displaced with respect to the central bore of the guiding element. This allows for the guiding element to be used by a user to rest at least one of the user's fingers when using the surgery instrument.

Preferably, the first end of the pivoting joint element is removably connected to the corresponding actuation arm, for ease of production.

Further advantageous embodiments according to the invention are described in the following claims.

It should be noted that the technical features described above or below may each on its own be embodied in an ophthalmic surgery instrument and/or in a method, i.e. isolated from the context in which it is described, separate from other features, or in combination with only a number of the other features described in the context in which it is disclosed. Each of these features may further be combined with any other feature disclosed, in any combination.

The invention will be further elucidated on the basis of exemplary embodiments which are represented in the drawings. The exemplary embodiments are given by way of non-limitative illustrations of the invention. In the drawings:

### BRIEF DESCRIPTION OF THE DRAWINGS

FIG. 1 shows a schematic cross sectional view of an ophthalmic surgery instrument according to the invention;

FIG. 2 shows a schematic cross sectional partial view of the ophthalmic surgery instrument shown in FIG. 1, in a compressed state;

FIG. 3 shows a schematic cross sectional partial view of the ophthalmic surgery instrument shown in FIG. 1, in a decompressed state;

FIG. 4 shows a first schematic perspective view of a pivoting joint element in the ophthalmic surgery instrument shown in FIG. 1;

FIG. 5 shows a second schematic perspective view of a pivoting joint element in the ophthalmic surgery instrument shown in FIG. 1;

FIG. 6 shows a third schematic perspective view of a pivoting joint element in the ophthalmic surgery instrument shown in FIG. 1, and

FIG. 7 shows a schematic perspective view of another ophthalmic surgery instrument according to the invention.

### DETAILED DESCRIPTION

In the figures identical or corresponding parts are represented with the same reference numerals. The drawings are only schematic representations of embodiments of the invention.

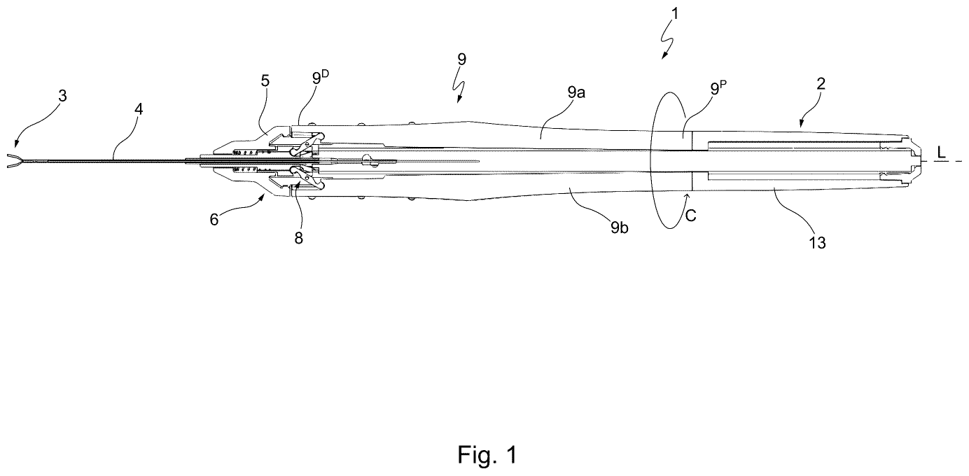

FIG. 1 shows a schematic cross sectional view of an ophthalmic surgery instrument 1 according to the invention. The instrument 1 includes a handling unit 2 having a longitudinal axis L. Further, the instrument 1 includes a surgical module 3 provided on the handling unit 2 and extending away therefrom along the longitudinal axis L thereof, and a shaft 4 that at least partially surrounds the surgical module 3. The surgical module 3 and the shaft 4 are mutually movable along the longitudinal axis L of the handling unit 2. In the shown embodiment, the shaft 4 moves along the longitudinal axis L relative to the handling unit 2, while the surgical module 3 remains mainly stationary along the longitudinal axis L relative to the handling unit 2.

The ophthalmic surgery instrument 1 also includes a guiding element 5 arranged at a distal end 6 of the handling unit 2, the guiding element 5 being provided with a central bore 7 that is traversed by the surgical module 3 and the shaft 4 extending from the handling unit 2 outwardly along the longitudinal axis L thereof.

The handling unit 2 further comprises an actuation unit 9 having a plurality of actuation arms 9a-d, also referred to as levers, arranged along the circumferential direction C around the longitudinal axis L of the handling unit 2. The actuation arms or levers 9, also referred to as basket, each have a proximal end 9P and a distal end 9D. Generally, the actuation arms 9 are bendable, and adjustable between a compressed state and a decompressed state, wherein the actuation arms 9 in the compressed state are closer to the longitudinal axis L of the handling unit 2 than in the decompressed state. Preferably, the actuation arms 9 are biased towards the decompressed state.

The actuation unit 9 further includes a proximal stationary element 13 that remains axially stationary relative to the handling unit 2.

The arm proximal ends 9P of the actuation arms are connected to the proximal stationary element 13, e.g. via respective intermediate hinges, while the arm distal ends 9D of the actuation arms 9 are free ends.

The handling unit 2 also comprises a driving unit 8 for driving, upon manual actuation of the actuation unit 9, the shaft 4 relative to the guiding element 5 back and forth through the central bore 7 of the guiding element 5.

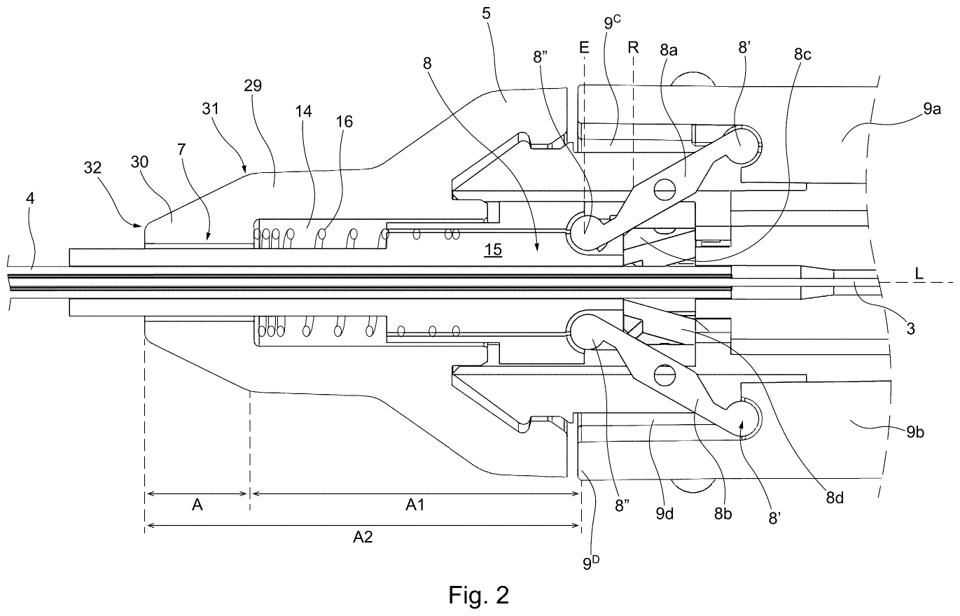

FIG. 2 shows a schematic cross sectional partial view of the ophthalmic surgery instrument 1 shown in FIG. 1, in a compressed state. In FIG. 2, the driving unit 8 is more visible than in FIG. 1.

As shown in FIG. 2, the driving unit 8 includes a radial-to-axial linkage that is constructed and arranged to convert a radial movement provided by the actuation unit 9 through the actuation arms 9 to axial movement of the shaft 4 with respect to the surgical module 3. The radial-to-axial linkage 9 has a set of pivoting joint elements 8a-d, also referred to as crank arms, each pivoting joint element 8a-d having a first end 8′ actuated by the actuation unit 9, and a second end 8″ associated with the shaft 4. The number of pivoting joint elements or crank arms 8 is the same as the number of actuation arms or levers 9, such that each actuation arm 9 actuates a corresponding pivoting joint element 8. As described in more detail below, the first ends 8′ of the pivoting joint elements 8 are pivotably connected to corresponding actuation arms 9, the actuation unit 9 directly driving the driving unit 8.

Further, in the shown embodiment, the guiding element 5 includes a cavity 14 aligned with the central bore 7, wherein the driving unit 8 further includes an annular holding element 15 received in the cavity 14 of the guiding element 5, the annular holding element 15 being fixedly attached to the shaft 4 and associated with the second ends 8″ of the pivoting joint elements or crank arms 8a-d.

Upon adjusting the actuation arms 9 from the decompressed state towards the compressed state, by manually compressing the actuation arms 9, the second end 8″ of the pivoting joint elements 8 moves along the longitudinal axis L of the handling unit 2, away from the proximal stationary element 13, and towards the guiding element 5, the result as shown in FIG. 2. As is also shown in FIG. 2, the second end 8″ of the pivoting joint elements 8 is arranged at a location that is proximally displaced with respect to the central bore 7, thereby allowing the guiding element 5 or parts of the guiding element to rest one or more of a user's fingers on while the instrument 1 is in use. In this process, the first ends 8′ of the pivoting joint elements 8 pivot with respect to the second ends 8″ towards the longitudinal axis L.

The instrument 1 further has a spring element 16 received in the cavity 14 for biasing the annular holding element 15 towards the handling unit 2. Similarly, the actuation arms 9 are biased towards the decompressed state. Then, the actuation arms 9 are in the decompressed state or are at least tending towards said decompressed state, when not manually actuated.

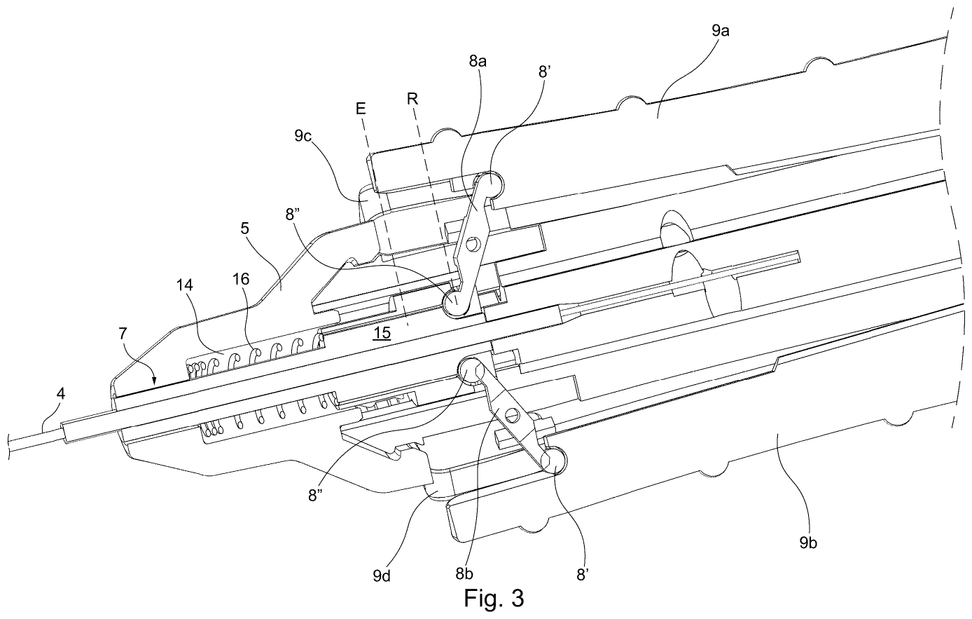

In the shown embodiment, refer in particular to FIG. 3, the number of visible actuation arms 9 is four, while the total number of actuation arms 9 of the actuation unit 9, around the longitudinal axis L of the handling unit 2, in the circumferential direction C, is eight. The number of actuation arms 9 may be more or less than eight. Preferably, the number of actuation arms 9 is even, such as four, six or eight. Then, the circumferential positions of the individual actuation arms can be chosen, preferably evenly distributed in the circumferential direction C, in such a manner that each actuation arm forms, together with another actuation arm positioned at circa 180 degrees in the circumferential direction C, a pair of opposing actuation arms 9. The actuation arms 9 can be formed as pairs of actuation arms. A user of the ophthalmic surgery instrument 1 may operate a pair of actuation arms by pressing said specific pair of actuation arms simultaneously. The multiple pairs of opposing actuation arms each have a specific circumferential orientation with respect to the surgical module. As an example, in the case of eight actuation arms, the user may select a specific a specific circumferential orientation of the arms 9 with respect to the surgical module 3, e.g. circa 0 degrees, circa 45 degrees, circa 90 degrees or circa 135 degrees in the circumferential direction C. Upon compressing the selected pair of actuation arms 9 the annular holding element 15 moves along the longitudinal axis L towards the guiding element 5, towards an extended axial position E. Alternatively, the actuation unit may have an uneven number of arms 9. Generally, the number of actuation arms 9 is at least three or four. This allows the user to hold the instrument in different orientations with the same grip. This is possible in case the number of actuation arms 9 is three. However, the case in which the number of actuation arms is at least four, is more preferable, because this allows for positioning for two of the actuation arms at opposite positions allowing a sure to more effectively compress.

FIG. 3 shows a schematic cross sectional partial view of the ophthalmic surgery instrument shown in FIG. 1, in a decompressed state.

Here, the second ends 8″ of the pivoting joint elements 8 are located in a rest axial position R along the longitudinal axis L. The first ends 8′ of the pivoting joint elements 8 are pivotably connected to the corresponding actuation arms 9, e.g. removably or permanently, so as to transfer both a pushing force from the actuation arm 9 and a pulling force from the pivoting joint element 8. The pushing force is applied by the actuation arm 9, upon manual actuation, moving the actuation arm 9 towards the compressed state. Then, the second ends 8″ of the pivoting joint elements are driven towards the guiding element 5, along the longitudinal axis L of the handling unit 2, and the annular holding element 15 moves away from the handling unit 2 pulling the second ends 8″ of all pivoting joint elements 8 also away from the handling unit 2. As a consequence, the first ends 8′ and the corresponding actuation arms 9 are pulled radially towards the longitudinal axis L, into the compressed state. Hence, the actuation arms 9 are mutually coupled and move as an ensemble when at least a subset of actuation arms 9 is manually compressed.

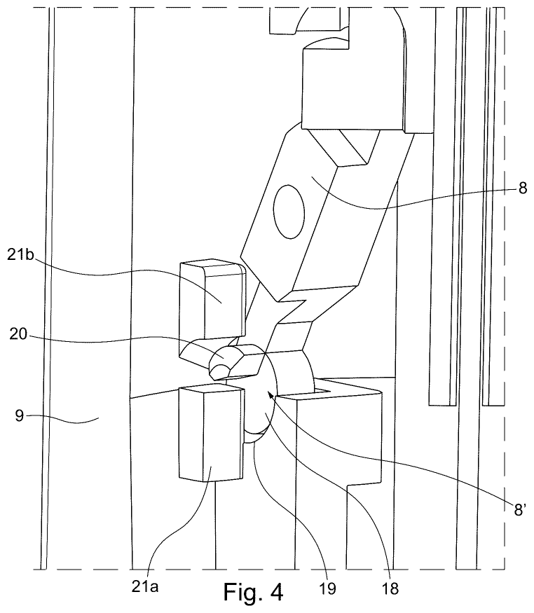

FIG. 4 shows a first schematic perspective view of a pivoting joint element 8 in the ophthalmic surgery instrument 1 shown in FIG. 1. Here, the first end 8′ of the pivoting joint element 8 is pivotably coupled to the corresponding actuation arm 9 via an axle bearing mechanism. The first end 8′ is mainly formed as a disc 18 received in a corresponding cavity 19 formed on the actuation arm 9. The first end 8′ further has a notch 20, and the actuation arm 9 has block shaped structures 21a,b locking the disc 18 in the cavity 19 when the pivoting joint element 8 is in operational use, i.e. swiveling in a limited angle range of operation. During assembly and de-assembly, the pivoting joint element 8 can be brought in an orientation outside said angle range of operation, for positioning and releasing, respectively, the first end 8′ from the actuation arm 9.

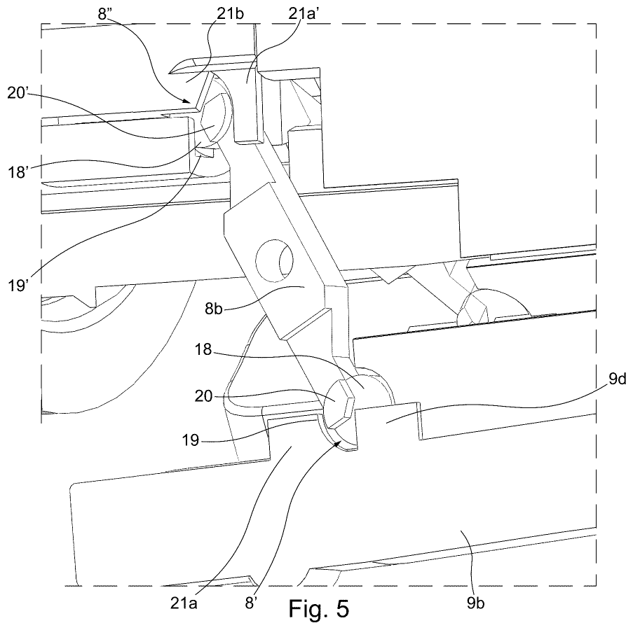

FIG. 5 shows a second schematic perspective view of a pivoting joint element in the ophthalmic surgery instrument shown in FIG. 1. Here, both the first end 8′ and the second end 8″ of the pivoting joint element 8 are shown. The pivoting connection structure of the second end 8″, to the annular holding element 15, is similar to the pivoting connection structure of the first end 8′ to the corresponding actuation arm 9, the second end 8″ having a similar disc 18′, notch 20′ and corresponding receiving cavity 19′ and blocking structures 21′a, b.

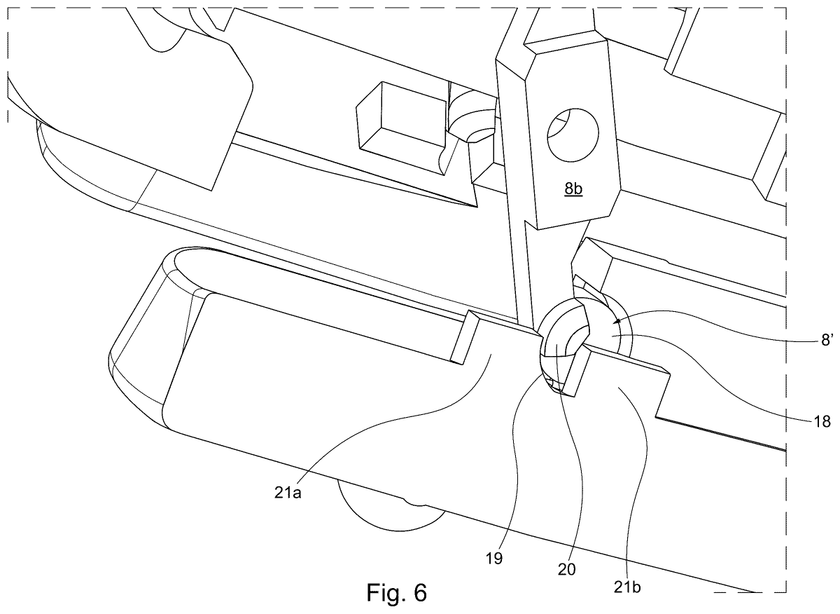

FIG. 6 shows a third schematic perspective view of a pivoting joint element 8 in the ophthalmic surgery instrument 1 shown in FIG. 1, wherein the first end 8′ of the pivoting joint element 8 is shown in more detail.

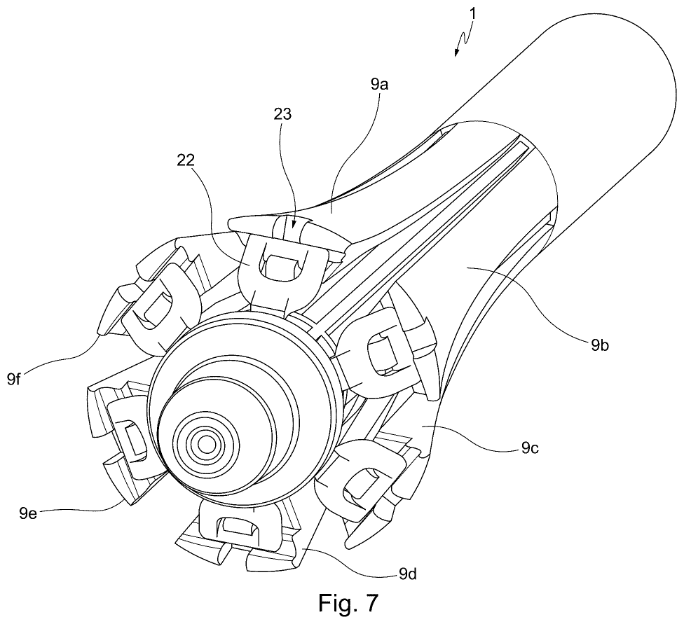

FIG. 7 shows a schematic perspective view of another ophthalmic surgery instrument 1 according to the invention. Here, the first end 8′ of the pivoting joint element 8 is connected to the corresponding actuation arm 9 via another axle bearing mechanism. The actuation unit has six actuation arms 9a-f that are connected to the first end 8′ of the corresponding pivoting joint elements 8. Each first end 8′ is provided with a ring-shaped element 22 that is bearingly and rotatably received and connectedin curved cavity 23 formed on the corresponding actuation arm 9 such that both a pulling force and a pushing force can be transferred through the hingeable connection.

It is noted that the cavity 19 or bearing can be formed on the actuation arm 9, as shown in the implementations illustrated in FIGS. 4-7, or alternatively, on the first end 8′ of the pivoting joint element 8. It is further noted that the pivotably connection of the first end 8′ to the actuation arm 9 can be implemented in another manner, e.g. using a hinge structure.

The actuation arms 9 are integrally formed. By providing the actuation arms 9 with portions having different stiffness, a bendable arm structure is realized. By pressing the arms radially inwardly, at least portions of the arms 9 bend radially inwardly. In an alternative embodiment, the actuation arms may each include at least two arm segments and at least a hinge 9″ interconnecting the at least two arm segments, the arm segments having a stiffness that is larger than a stiffness of the hinge. The arm segments can be made separately and assembled to each other, via the hinge, or, alternatively, can be made as an integral part having locally different stiffness values. In principle, further arm segments can be added to the two segment arm structure, e.g. via an additional hinge. A local stiffness can be set by making the arm thinner or thicker and/or by changing its material. As a further alternative, the actuation arm can be formed without hinge, however preferably such that the actuation arm bends upon manual actuation.

The actuation arms 9 can be designed in a way to provide different material properties to set the haptic conditions of the mechanism.

Further, the actuation arms 9 or basket can be personalized for instance as a 2 k injected part with variable and different hardness and/or flexibility of the at least 2 arm segments 9′, 9″.

In the shown embodiment, the guiding element 5 is stationary relative to the handling unit 2.

As discussed, the ensemble of the actuation unit 9 and the driving unit 8 are arranged for driving the shaft 4, in particular for driving the shaft 4 away from the handling unit 2 upon manual actuation of the actuation unit 9. Here, the surgery unit 3 may be axially stationary relative to the handling unit 2. It is noted however, that, in principle, the ensemble of the actuation unit 9 and the driving unit 8 may be arranged for moving the surgery unit 3 along the longitudinal axis L, while the shaft 4 remains axially stationary relative to the handling unit 2. Then, the driving unit 8 may be axially coupled, directly or indirectly, with the surgery unit 3.

In the shown embodiment, in particular referring to FIG. 2, the guiding element 5 is provided with a nosecone 29 comprising at least one cone-shaped portion 30 or truncated cone-shaped portion having a proximal end 31 and a distal end 32, wherein the nosecone is located at a distal position with respect to the actuation arms 9.

In a particular embodiment, in a decompressed state, a distal end 9D of at least one of the actuation arms 9 is located at a first axial distance A1 from the proximal end 31 of the cone-shaped part 30 of the nosecone 29 between about 8 mm and about 16 mm. Further, the distal end 9D of at least one of the actuation arms 9 may be located at a second axial distance A2 from the distal end 32 of the cone-shaped part 30 of the nosecone 29 between about 12 mm and about 25 mm. Preferably, the cone-shaped portion 30 of the nosecone 29 has an axial length A of between about 2 mm and about 10 mm.

The surgical module 3 is formed as an integral part or as an assemblage of separate parts. The surgical module 3 may have a rod that is provided, at a distal end thereof, with an operating unit, such as a forceps, pair of scissors, fibre, scraper, vertical scissor or fiber assembly. At least one the actuation arms 9 of the actuation unit may have an outer or exterior contour deviating from an outer or exterior contour of other actuation arms, such that the at least one actuation arm is aligned with a circumferential orientation of the operating unit of the surgical module 3, e.g. the orientation of the forceps. By moving the shaft 4 relative to the surgical module 3, the distal end can selectively be exposed or opened so as to actuate or operate the operating unit, e.g. for grabbing and/or releasing tissue.

The invention is not restricted to the embodiments described herein. It will be understood that many variants are possible.

These and other embodiments will be apparent for the person skilled in the art and are considered to fall within the scope of the invention as defined in the following claims. For the purpose of clarity and a concise description features are described herein as part of the same or separate embodiments. However, it will be appreciated that the scope of the invention may include embodiments having combinations of all or some of the features described.

1. An ophthalmic surgery instrument, comprising:
a handling unit having a longitudinal axis,
a surgical module provided on the handling unit and extending away therefrom along the longitudinal axis thereof, and
a shaft at least partially surrounding the surgical module,
wherein the surgical module and the shaft are mutually movable relative along the longitudinal axis of the handling unit,
a guiding element arranged at a distal end of the handling unit, the guiding element being provided with a central bore that is traversed by the surgical module and the shaft extending from the handling unit outwardly along the longitudinal axis thereof, wherein the handling unit comprises
an actuation unit comprising a plurality of actuation arms arranged around the longitudinal axis of the handling unit, and
a driving unit arranged to, upon manual actuation of the actuation unit, drive the shaft relative to the guiding element back and forth through the central bore of the guiding element, the driving unit including a radial-to-axial linkage constructed and arranged to convert a radial movement provided by the actuation unit through the actuation arms to axial movement of the shaft with respect to the surgical module, wherein the radial-to-axial linkage comprises a set of pivoting joint elements, each pivoting joint element having a first end and a second end, said second end being associated with the shaft,
wherein the number of pivoting joint elements corresponds with the number of actuation arms, and
wherein the first end of each pivoting joint element is pivotably connected to a corresponding actuation arm.

2. An ophthalmic surgery instrument according to claim 1, wherein the first end of the pivoting joint element is removably connected to the corresponding actuation arm.

3. An ophthalmic surgery instrument according to claim 1, wherein the first end of the pivoting joint element is connected to the corresponding actuation arm so as to transfer both a pushing force from the actuation arm and a pulling force from the pivoting joint element.

4. An ophthalmic surgery instrument according to claim 1, wherein the first end of the pivoting joint element is connected to the corresponding actuation arm via an axle bearing mechanism.

5. An ophthalmic surgery instrument according to claim 1, wherein the first end of the pivoting joint element is connected to the corresponding actuation arm via a hinge structure.

6. An ophthalmic surgery instrument according to claim 1, wherein the actuation arms are adjustable between a compressed state and a decompressed state, wherein the actuation arms in the compressed state are closer to the longitudinal axis of the handling unit than in the decompressed state.

7. An ophthalmic surgery instrument according to claim 1, wherein the actuation arms are biased towards the decompressed state.

8. An ophthalmic surgery instrument according to claim 1, wherein the number of actuation arms is three or more, or wherein the number of actuation arms is four or more.

9. An ophthalmic surgery instrument according to claim 1, wherein the number of actuation arms is even, preferably 4, 6 or 8.

10. An ophthalmic surgery instrument according to claim 1, wherein the pivoting joint element is arranged such that its second end moves along the longitudinal axis of the handling unit upon adjusting the actuation arms from the decompressed state towards the compressed state.

11. An ophthalmic surgery instrument according to claim 1, wherein, in the decompressed state, the second ends of the pivoting joint elements are located in a rest axial position along the longitudinal axis of the handling unit, and wherein, in the compressed state, the second ends of the pivoting joint elements are driven towards the guiding element.

12. An ophthalmic surgery instrument according to claim 1, wherein the actuation unit directly actuates the driving unit.

13. An ophthalmic surgery instrument according to claim 1, wherein the guiding element is substantially axially stationary relative to the handling unit.

14. An ophthalmic surgery instrument according to claim 1, wherein the guiding element includes a cavity aligned with the central bore, and wherein the driving unit further includes an annular holding element received in the cavity of the guiding element, the annular holding element being fixedly attached to the shaft and associated with the second ends of the pivoting joint elements.

15. An ophthalmic surgery instrument according to claim 1, wherein the actuation unit and the driving unit are arranged for driving the shaft, in particular for driving the shaft away from the handling unit upon manual actuation of the actuation unit.

16. An ophthalmic surgery instrument according to claim 1, wherein the guiding element is provided with a nosecone comprising at least one cone-shaped portion having a proximal end and a distal end, wherein the nosecone is located at a distal position with respect to the actuation arms.

17. An ophthalmic surgery instrument according claim 16, wherein a distal end of at least one of the actuation arms is located at an axial distance from the proximal end of the cone-shaped part of the nosecone between about 8 mm and about 16 mm and/or at an axial distance from the distal end of the cone-shaped part of the nosecone between about 12mm and about 25mm.

18. An ophthalmic surgery instrument according to claim 16, wherein the cone-shaped portion of the nosecone has an axial length of between about 2 mm and about 10 mm.
